(19) United States
(12) Patent Application Publication
(10) Pub. No.: US 2026/0033935 A1
(43) Pub. Date: Feb. 5, 2026

(54) PET HEALTH AND WELLNESS SYSTEM

(71) Applicant: VETINSTANT HEALTHCARE PRIVATE LIMITED, Chennai (IN)

(72) Inventor: Vivek Srinivas Paravastu, Chennai (IN)

(73) Assignee: VETINSTANT HEALTHCARE PRIVATE LIMITED, Chennai, TN (IN)

(21) Appl. No.: 18/997,036

(22) PCT Filed: Jan. 18, 2023
(86) PCT No.: PCT/IN2023/050057

§ 371 Date: Jan. 18, 2025

(30) Foreign Application Priority Data
Oct. 7, 2022 (IN) 202241057598

(51) Int. Cl. A61D 13/00; A61D 99/00

(52) U.S. Cl. CPC A61D 13/00; A61D 99/00

(57) ABSTRACT

Described herein is a pet health and wellness system for performing medical examination of an animal, such as a pet, by using a hand-held diagnostic device in a non-invasive manner from the comfort of their homes. A feedback system and a stencil are provided to ensure that the vitals, such as core body temperature, record heart and lung auscultations as well as measure pulse and oxygen saturation, acquired by the device are accurate. The system includes a portable examination device comprising one or more examination units for obtaining and recording essential vitals of the animal by a user and a processing unit for storing and managing data corresponding to the essential vitals and medical information of the animal.

### TECHNICAL FIELD

The present subject matter in general relates to health and wellness system for animals and in particular relates to a device, system and method of performing one or more medical examinations of an animal by accurately acquiring the medical data with the help of a diagnostic device.

### BACKGROUND

Raising a pet is a popular way of life almost anywhere in the world. Most animals ranging from dogs, cats, and even some wilder breeds of animals are found to be friendly enough to be considered as pets. That being said, maintaining the health of a pet is a cumbersome task. Generally, for any medical examination or diagnosis including routine check-ups, people have to take their pets and other domesticated animals to the veterinary clinics or hospitals.

However, a visit to a clinic might not always be possible and sometimes it would be more comfortable for the pets to be treated at home. Also, there is a lot of stress which pets undergo at the clinic, which makes it difficult for the veterinarians to conduct a thorough examination. Further, there can be situations, where pet parents are required to do a medical examination of the pet themselves either as a routine check-up or due to sickness of the pet. However, majority of diagnostic devices known in the art are invasive and can only be handled by a trained medical professional. Moreover, there is no system known in the art that enables pet parents to examine pets at the comfort of homes.

In view of the above, a heretofore unaddressed need exists in the industry to address the aforementioned deficiencies and inadequacies of the existing diagnostic systems.

### SUMMARY

The following presents a simplified summary of the subject matter in order to provide a basic understanding of some of the aspects of subject matter embodiments. This summary is not an extensive overview of the subject matter. It is not intended to identify key/critical elements of the embodiments or to delineate the scope of the subject matter. Its sole purpose to present some concepts of the subject matter in a simplified form as a prelude to the more detailed description that is presented later.

The present subject matter provides a device, system and method of performing one or more medical examinations of an animal, such as a pet, by using a diagnostic device. Each aspect of the medical examination according to the present subject matter can be provided with an assistance tool or instructions on the placement and navigation to accurately acquire the medical data in an embodiment.

According to a preferred embodiment, the present invention comprises a hand-held diagnostics device, which is configured to assist a pet owner to perform certain aspects of pet examination from the comfort of their homes in a non-invasive manner. As the present device is to be used by the pet owner at home, it is ensured that the same is non-invasive, assists in correct placements, has some pre-determined standards like length of recording and quality of the recording/data, has a feedback system to confirm that the readings are taken correctly, and is capable of connecting or sending the data to a trained veterinarian to interpret and diagnose the result.

The device according to the present subject matter is configured to measure the core body temperature of the pet, record heart and lung auscultations as well as measure pulse and oxygen saturation. In a preferred embodiment, the present device comprises a silicon stencil to correctly position and take heart and lung auscultations at different positions. The present device is also provided with a feedback system that informs the pet owner, whether the recording or the data taken is correct or prompt him/her to re-take the recording. In an embodiment, the present device is configured to connect the pet owner with his/her own vet and share the examination data with the vet.

In the present device, different aspects of vitals measurement are combined into one integrated unit. The sound from heart and lung area of the pet is sensed by the acoustic sensors. The motion sensors evaluate the accuracy of the readings as well as give feedback and guidance to the owner for accurate measurement. In an embodiment, the motion sensors are spatial motion sensors that comprise a processing engine to measure change of position of the pet over time to yield physical parameters. In a preferred embodiment, the temperature sensors comprise one of infrared (IR) temperature sensors to calculate the body temperature of the pet, wherein the temperature sensor is to be placed on the anal verge area. The anal verge region is kept under laser focus of the IR temperature sensor for thermal detection, and the IR temperature sensor is in contact with the skin of the pet on a specific area where there is no fur and reflects the body temperature of the pet.

According to an embodiment, the acoustic sensors comprise non-contact reflective acoustic units that are configured to cancel external noise while recording the heart and lung sounds. A filtering circuitry to capture heart and lung auscultations of the pet is also provided in the system according to the present subject matter.

In a preferred embodiment, the device according to the present subject matter is a hand-held animal vital examination device that helps in acquisition and diagnosis of medical data by assisting and guiding the owner in taking the vital parameters of pets for better and objective clinical decision making. According to an embodiment, the parameters monitored include body temperature, heart auscultations, lung auscultations, pulse and oxygen saturation.

The present subject matter also provides a pet health and wellness system that employs Internet of Things (IoT) technology for collecting the data in a central location and can be used by different health specialists to alert the owners as well as to ensure preventive treatments.

The system according to the present subject matter has unique design elements. In an embodiment, the system includes a specifically designed and calibrated optical probe system to measure temperature. The system further comprises a noise handling mechanism to get pure data for the HR, a specially designed and calibrated oxygen saturation and pulse measurement extension, a system enclosure to make it an integrated device, a separate stencil made of silicone or any other material for correction placement and positioning of the device, and a feedback system by way of tracking motion artifacts and external noise by way of analysing the vibration and movement of the device and by processing the acquired data against a referenced data set. According to the present subject matter, the medical data acquisition guidance includes one or more of an indication of a check to be performed, navigational instructions, operational instructions, positioning instructions, and examination process instructions. A verification process is also provided that verifies whether the acquired data meets pre-defined standards or not. In an embodiment, the pre-defined standards include one or more of the required length of reading, recorded sound volume, and reading parameters thresholds. According to the present subject matter, where a respective pre-defined standard is not met or the processed data in comparison with the reference data is false, the user is prompted to retake the examination and also feedback is given with reference to the reason for re-take. The pet health and wellness system also includes a software that offers two different interfaces to the health specialists and the owners, i.e., (i) for the veterinarians, the software offers real time data about the pet on all the vital parameters and helps the vets to decide the course of action, (ii) for the owners, the software offers the generic health of the pet with an alert system to enable the owners to connect with qualified professional who can then do an initial diagnosis at home.

### BRIEF DESCRIPTION OF ACCOMPANYING DRAWINGS

The present invention, both as to its organization and manner of operation, together with further objects and advantages, may best be understood by reference to the following description, taken in connection with the accompanying drawings. These and other details of the present invention will be described in connection with the accompanying drawings, which are furnished only by way of illustration and not in limitation of the invention, and in which drawings:

FIG. 1 illustrates a flow diagram depicting a process of operating a pet health and wellness system where a user initiates with the registration on a mobile App according to an embodiment of the present subject matter.

FIG. 2 illustrates a flow diagram depicting a process of operating the pet health and wellness system during pet examination process in an embodiment of the present subject matter.

FIG. 3 illustrates a block diagram of a pet diagnostic device in accordance with an embodiment of the present subject matter.

FIG. 4 illustrates a system architecture of the pet health and wellness system in accordance with an embodiment of the present subject matter.

FIG. 5 illustrates the pet health and wellness system depicting its various modules in accordance with an embodiment of the present subject matter.

FIGS. 6 to 10 illustrate different perspective views of the pet diagnostic device in accordance with an embodiment of the present subject matter.

FIG. 11 illustrates a front view of a pair of silicon stencils in accordance with an embodiment of the present subject matter.

FIG. 12 illustrates the pair of silicon stencils mounted on a pet in accordance with an embodiment of the present subject matter.

The above mentioned drawings are illustrative of particular examples for enabling systems and methods of the present disclosure, are descriptive of some of the methods and mechanism, and are not intended to limit the scope of the invention. The drawings are not to scale (unless so stated) and are intended for use in conjunction with the explanations in the following detailed description.

### DETAILED DESCRIPTION

The following presents a detailed description of various embodiments of the present subject matter with reference to the accompanying drawings.

The embodiments of the present subject matter are described in detail with reference to the accompanying drawings. However, the present subject matter is not limited to these embodiments which are only provided to explain more clearly the present subject matter to a person skilled in the art of the present disclosure. In the accompanying drawings, like reference numerals are used to indicate like components.

The specification may refer to “an”, “one”, “different” or “some” embodiment(s) in several locations. This does not necessarily imply that each such reference is to the same embodiment(s), or that the feature only applies to a single embodiment. Single features of different embodiments may also be combined to provide other embodiments.

As used herein, the singular forms “a”, “an” and “the” are intended to include the plural forms as well, unless expressly stated otherwise. It will be further understood that the terms “includes”, “comprises”, “including” and/or “comprising” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “attached” or “connected” or “coupled” or “mounted” to another element, it can be directly attached or connected or coupled to the other element or intervening elements may be present. As used herein, the term “and/or” includes any and all combinations and arrangements of one or more of the associated listed items.

The figures depict a simplified structure only showing some elements and functional entities, all being logical units whose implementation may differ from what is shown.

The present subject matter provides a system, a device and a method of performing one or more medical examinations of animals, particularly pets and other domesticated animals. The device according to the present invention is a hand-held diagnostic device, also referred to as an integrated medical examination device, which guides the pet owners to obtain the vital parameters of pets at home. The diagnostic device assists the pet owners through references as well as through a stencil, preferably made of silicone or any other material, to accurately place the device at the apt location on the pet for accurate measurement of vitals. The present device also analyses the readings with a referenced set and gives feedback to the owner on the quality of the measurements/medical data acquisition. A plurality of data acquisition devices comprising various sensors, measurement hardware, and controller with programmable software are also provided in the system to acquire data.

In a preferred embodiment, the integrated medical examination device is configured to record and measure heart and lung auscultations, take temperature readings from the anal verge area non-invasively and take oxygenation as well as pulse readings in active animals from their lips. The integrated medical examination device comprises a temperature module that is configured so that it rests accurately on the wall of the anal region of the pet. The integrated medical examination device also comprises an SPO2 acquisition peripheral that is configured such that SPO2 readings can be taken in animals in a predetermined area. The stencil of the integrated medical examination device is preferably made of silicone to guide the user in correct positioning of the cardiac and lung auscultation points of the integrated medical examination device in an embodiment. However, in another embodiment, the stencil can be made from any other material. During operation, the stencil is placed at the first teat of the pet animal, which is considered as the reference point. The integrated medical examination device is configured to give guidance as well as feedback to the user or pet owner by way of a mobile App, thereby enabling the users to satisfy themselves as to the accuracy of the readings.

Accordingly, a health and wellness system for an animal, such as a pet, is provided. The system according to the present invention comprises a portable examination device configured to perform one or more aspects of examination of the animal in a non-invasive manner. The portable examination device comprises one or more examination units for obtaining and recording essential vitals of the animal by a user. The system further comprises a processing unit for storing and managing data corresponding to the essential vitals and medical information of the animal.

In an embodiment, the essential vitals comprise one or more of core body temperature of the pet, heart auscultations, lung auscultations as well as pulse and oxygen saturation.

In another embodiment, the health and wellness system is a hand-held device.

In yet another embodiment, the health and wellness system further comprises a silicon stencil to correctly position the portable examination device on the body of the pet and obtain heart and lung auscultations at different positions.

In yet another embodiment, the health and wellness system further comprises a feedback system for providing feedback about the correctness of the data acquired by the portable examination device to the user.

In yet another embodiment, the portable examination device is configured to connect the user with a medical professional.

In yet another embodiment, the one or more examination units of the portable examination device comprise one or more heat based sensors, one or more acoustic sensors, one or more photo optic sensors and one or more motion sensors.

In yet another embodiment, the one or more acoustic sensors are mounted on a first wall of the portable examination device and is configured to sense sound from heart and lung area of the animal.

In yet another embodiment, the acoustic sensors comprise non-contact reflective acoustic units that are configured to cancel external noise while recording the heart and lung sounds.

In yet another embodiment, the one or more heat based sensors comprise temperature sensors having one of IR temperature sensors to calculate the body temperature of the animal.

In yet another embodiment, the one or more temperature sensors are mounted on the side wall of the portable examination device and are configured to obtain temperature reading of the animal merely by getting in contact with the anal verge area of the animal.

In yet another embodiment, the one or more photo optic sensors comprise a foldable SPO2 measurement unit having a foldable SPO2 measurement member for measuring SPO2 readings of the animal merely by contacting the foldable SPO2 measurement member with one of the lips of the animal.

In yet another embodiment, the one or more motion sensors are configured to evaluate the accuracy of the readings and to give feedback and guidance to the user for accurate measurement.

In yet another embodiment, the one or more motion sensors comprise spatial motion sensors having a processing engine to measure change of position of the animal over time to yield physical parameters.

In yet another embodiment, the pet health and wellness system further comprises a filtering circuitry to capture heart and lung auscultations of the animal.

In yet another embodiment, the pet health and wellness system further comprises a noise handling mechanism to obtain pure data for the heart rate.

In yet another embodiment, the processing unit comprises a pet data repository for storing data corresponding to the essential vital parameters of the animal recorded by the portable examination device, storing medical history and other related information of the animal, and a management system for managing the data of the animal.

In yet another embodiment, the processing unit further comprises a pet owner module and a veterinarian module connected with each other.

In yet another embodiment, the pet owner module comprises a memory unit and a processor embedded in the medical examination device.

In yet another embodiment, each of the pet owner module and the veterinarian module comprises a video streaming module for enabling video streaming of the user with the medical professional.

In yet another embodiment, the veterinarian module comprises a trained reference medical data repository.

In yet another embodiment, the processing unit comprises a first interface for the user that provides the generic health of the pet with an alert system to enable the user to connect with the medical professional and a second interface for the medical professional that provides real time data about the animal on all the vital parameters.

In an embodiment, a health and wellness system for an animal, such as a pet, is provided that comprises an examination module configured to capture sensor data corresponding to the essential vitals of the animal from one or more sensors, a pet data repository for storing the sensor data, a microcontroller configured to obtain and the sensor data as input and process said sensor data, and a wireless fidelity based high speed radio module configured for direct data communication with a remote cloud server.

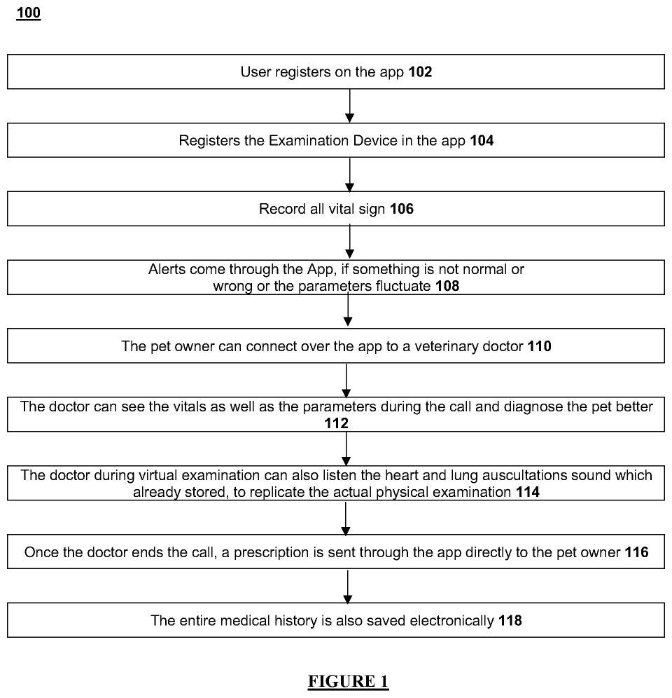

FIG. 1 illustrates a flow diagram depicting a process 100 of operating a pet health and wellness system where a user initiates with the registration on a mobile App according to an embodiment of the present subject matter. This figure broadly depicts the process or journey of the user for the system as a whole. As shown herein, the operation starts with the registration process 102 in which the user registers himself/herself. This step is followed by registration 104 of the integrated medical examination device in a mobile App. Upon registration, all the vitals of the pet animal are recorded 106 with the help of the integrated medical examination device. If the vitals are not normal or wrong or the parameters fluctuate, the alerts are indicated 108 in the mobile App, in which case the pet owner can connect 110 over the mobile App to a veterinary doctor. When connected, the doctor can view 112 the recorded vitals as well as the parameters in the call and diagnose the pet better. The doctor, during virtual examination, can also listen 114 the heart and lung auscultations sound, which is stored in the system while recording by the integrated medical examination device, to replicate the actual physical examination. Once the doctor ends the call, a prescription is sent 116 through the mobile App directly to the pet owner.

The entire medical history is also saved electronically in the system 118.

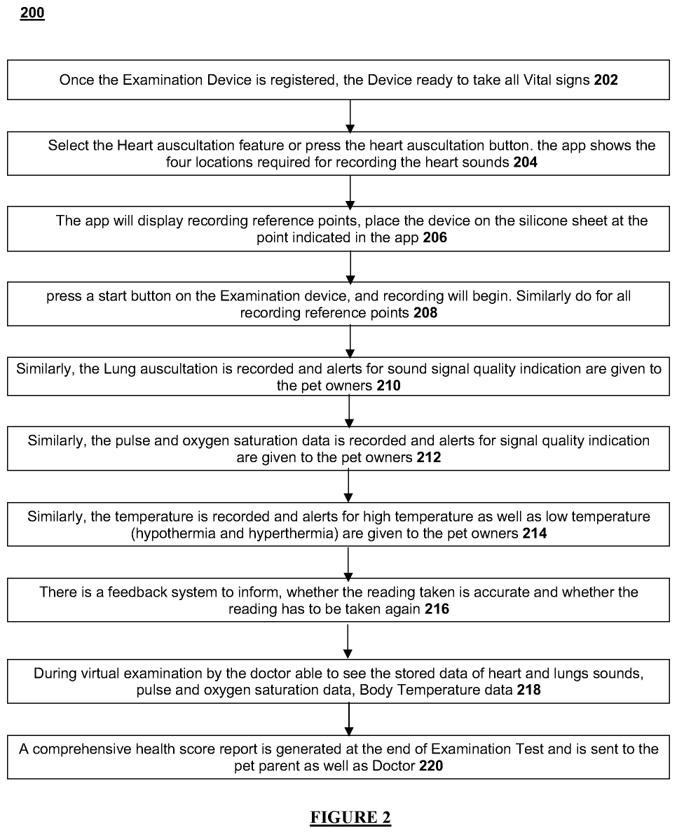

FIG. 2 illustrates a flow diagram depicting a process 200 of operating the pet health and wellness system during pet examination process in an embodiment of the present subject matter. This figure depicts the detailed flow/user process for the integrated medical examination device. Once the integrated medical examination device is registered, the device is ready 202 to take all vital signs of the pet. To take the vital signs, the user/pet owner selects 204 the heart auscultation feature or presses the heart auscultation button. In an embodiment, the mobile App shows the four locations required for recording the heart sounds. The mobile App displays 206 recording reference points. In this step, the user then places the device on the silicone sheet worn by the pet at the point indicated in the mobile App. The user presses 208 the start button on the examination device. After pressing a start button, recording of the vital signs corresponding to heart beat begins at that reference point in this step. Similarly, vital signs of all reference points are recorded by the user in this step. Similarly, the lung auscultation is recorded and alerts for sound signal quality indication are given to the user in step 210. In step 212, the pulse and oxygen saturation data is recorded and alerts for signal quality indication are given to the user. In step 214, the temperature is recorded and alerts for high temperature as well as low temperature (hypothermia and hyperthermia) are given to the user.

In a preferred embodiment, the present system includes a feedback system that indicates in step 216 whether the reading taken is accurate or inaccurate and whether it is required to take the reading again. During virtual examination, the doctor is able to see the stored data of heart and lungs sounds, pulse and oxygen saturation data as well as body temperature data in step 218. In a preferred embodiment, a comprehensive health score report is generated in step 220 at the end of examination test and is sent to the pet owner as well as the doctor. A pet owner at home is not experienced or trained to correctly measure the parameters at one go. Feedback at the time of taking the examination becomes important for the pet owner to know whether he has taken the reading correctly. The readings can go wrong either due to motion artifacts or external noise or wrong placement. The present device gives feedback on account of motion artifacts and external noise by way of analyzing the vibration and movement of the device and by processing the data.

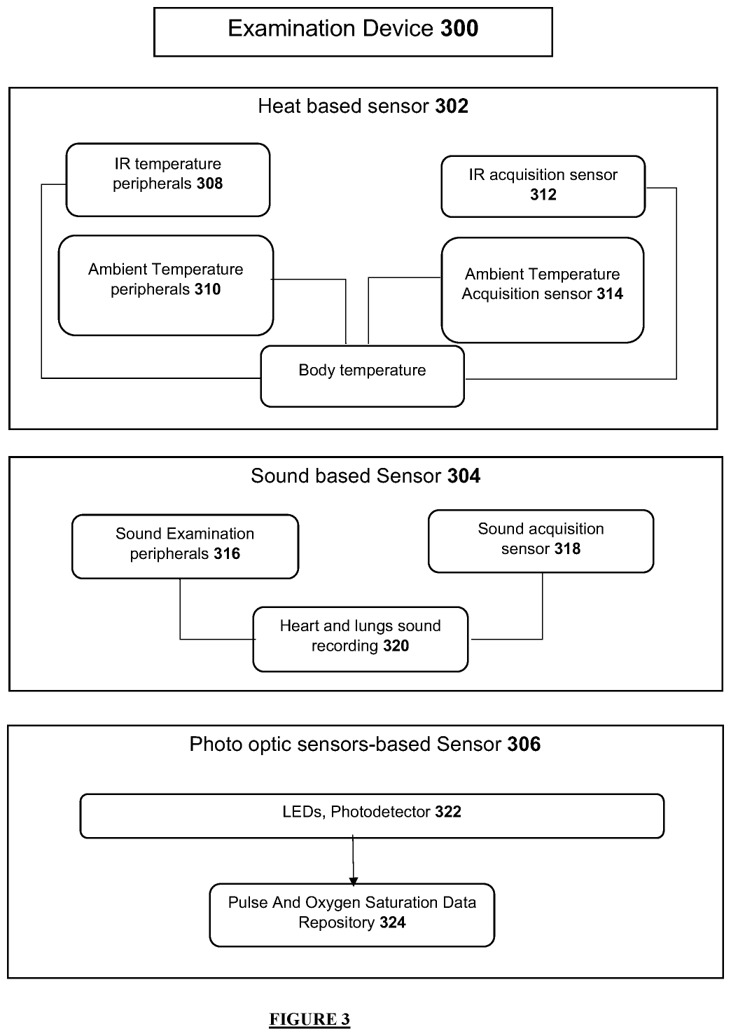

FIG. 3 illustrates a block diagram of a pet diagnostic device or an integrated medical examination device 300 in accordance with an embodiment of the present subject matter. As shown herein, the integrated medical examination device 300 comprises one or more heat based sensors 302 for measuring body temperature of the pet animal, one or more sound based sensors 304 for recording sound of heart and lungs and one of more photo-optic sensors 306 for recording pulse and oxygen saturation data. In an embodiment, the heat based sensor 302 comprises one or more infrared (IR) temperature peripherals 308, one or more relative humidity and ambient temperature peripherals 310, one or more infrared (IR) acquisition sensors 312 and one or more relative humidity and ambient temperature acquisition sensor 314. In a preferred embodiment, the IR temperature peripherals 308 can include, inter alia, various components that enable easy fit, comfortable adjustment and safe access. Such components can be, for example, made of plastic, rubber, etc. but are not limited to these materials. The IR acquisition sensors 312 can be, for example, a temperature detection sensor, or any other device capable of acquiring the body temperature at that specific part of the pet's body. In an embodiment, the IR acquisition sensors 312 may further be calibrated and modified to suit veterinary use. The relative humidity and ambient temperature peripherals 310 can include, inter alia, various components that enable easy fit, comfortable adjustment and safe access. Such components can be, for example, made of plastic, rubber, etc. but are not limited to these materials. The relative humidity and ambient temperature sensors 314 can be, for example, a temperature detection sensor, or any other device capable of acquiring the environmental temperature as well as relative humidity in the location where the pet is present. The relative humidity and ambient temperature acquisition sensors 314 further can be calibrated and modified to detect changes in the environmental temperature to develop a context in which the pet is present. By a combination of the IR temperature peripherals 308 and the IR temperature acquisition sensors 312, the relative humidity and ambient temperature peripherals 310, and the relative humidity and ambient temperature acquisition sensors 314, which are calibrated to a specific pet and normalization, body temperature of the pet is derived.

In an embodiment, the sound based sensor 304 comprises one or more sound examination peripherals 316 and one or more sound acquisition sensors 318. The sound acquisition sensors 318 can be, for example, a microphone, or any other device capable of acquiring sound data. In an embodiment, the sound acquisition sensors 318 can fit multiple sound frequencies that can be adjusted to fit recording of specific organ sound as, for example, heart sound frequencies are different than lung sound frequencies. The sound acquisition sensors 318, can also include various abilities to assist acquiring a quality sound such as noise cancellation filters, etc.

In an embodiment, the sound examination peripherals 316 can include, inter alia, various components that enable easy fit, comfortable adjustment and safe access. Such components can be, for example, made of plastic, rubber, etc. but are not limited to these materials. The sound acquisition sensors 304 can also include various abilities to assist acquiring a quality sound such as noise cancellation filters, etc. By a combination of the sound examination peripherals 316 and the sound acquisition sensors 318 including usage of active noise filtration techniques and adaptive filtering, heart and lung sound recording are transmitted and stored. In an embodiment, a storage module 320 is provided in the sound acquisition sensors 304 for storing the vitals of pet obtained by the present device.

In an embodiment, the photo-optic sensor 306 comprises one or more LEDs and photodetectors 322. In a preferred embodiment, the photo-optic sensor 306 includes a pulse and oxygen saturation data repository 324 in which the vitals of the pet obtained by the present device are stored. Motion based sensor module includes inter alia detection of movement and orientation of the examination device at any point of time.

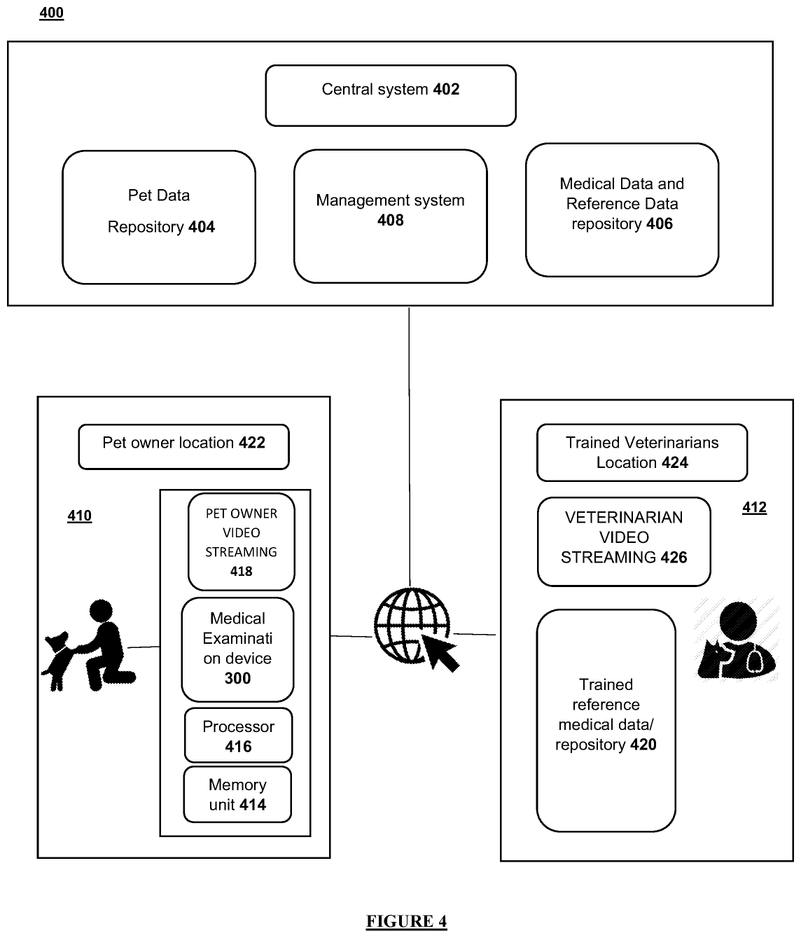

FIG. 4 illustrates a system architecture of the pet health and wellness system 400 in accordance with an embodiment of the present subject matter. The system 400 according to the present subject matter includes a processing unit or central sub-system 402 comprising a pet data repository 404 in which the information about the pet is stored, a medical data and reference data repository 406 in which the information about the medical history and other related information is stored, and a management system 408 that is configured to manage the data stored in the system. The central sub-system 402 is configured to connect a pet owner module 410 with a veterinarian module 412. In an embodiment, the pet owner module 410 comprises a memory unit 414 and a processor 416 that is embedded in the medical examination device 300. The pet owner module 410 also includes a video streaming module 418 for enabling video streaming to connect to the veterinarian via the veterinarian module 412. In an embodiment, the veterinarian module 412 comprises a trained reference medical data repository 420.

In FIG. 4, the pet owner location 422 represents the actual location of the pet owner, which may be away from the trained veterinarian location 424. The pet owner location 422 can be the pet owner's home or any other location. In an embodiment, the pet owner video streaming module 418 can be a mobile phone or any separate device attached to the mobile phone or connected to the mobile phone of the pet owner, which helps in streaming live videos of the pet's condition for examination. In another embodiment, the pet owner video streaming module 418 may be any other electronic device that enables streaming of live videos. In an embodiment, the processor 416 comprises digital signal processor (DSP), a microcontroller, a field programmable gate array (FPGA), an application specific integrated circuit (ASIC), etc. In an embodiment, the processor 416 is configured to receive instructions and control the components and operations of all the devices. In an embodiment, the memory unit 414 comprises ROM, hard disk, Flash memory etc.

Further, in FIG. 4, the trained veterinarian location 414 can be the veterinarian's clinic or any other location from which the veterinarian generally operates. The veterinarian module 412 includes the veterinarian video streaming module 426 that is configured to perform video streaming on veterinarian video streaming devices, such as mobile phones, desktop, laptop or any other internet connected device, from which the veterinarian can take video calls. When the transmitted data from the pet owner is received by the trained veterinarian, the data can be saved in the trained personnel data repository 420. In an embodiment, the data may include but is not limited to the data, sound files, video files as well as general medical reference and image detection data.

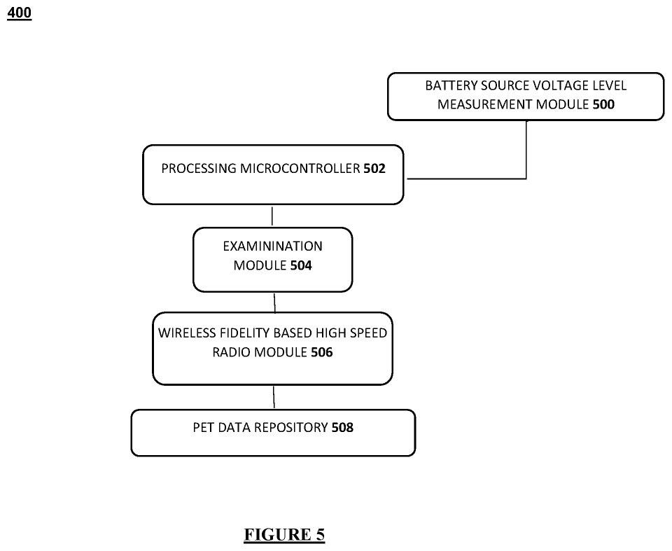

FIG. 5 illustrates pet health and wellness system 400 depicting its various modules in accordance with an embodiment of the present subject matter. As shown herein, the system 400 includes a battery source voltage level measurement module 500, a processing microcontroller 502, an examination module 504, a wireless fidelity based high speed radio module 506 and a pet data repository 508. In an embodiment, the battery source voltage level measurement module 500 is configured to measure the voltage level of the battery source. In an embodiment, the processing microcontroller 502 is configured to collect all the sensor data as input and process this data. The sensor readings are captured by using the examination module 504. These readings are transmitted via a common gateway module and then stored in a pet data repository 508. In the embodiment depicted in FIG. 5, the pet data repository 508 comprises individual pet data repository that is a data storage system configured to store various data relating to the individual pets acquired out of the data acquisition sensors and various medical data acquired during medical examination. The wireless fidelity based high speed radio module 506 is configured for direct data communication with a remote cloud server.

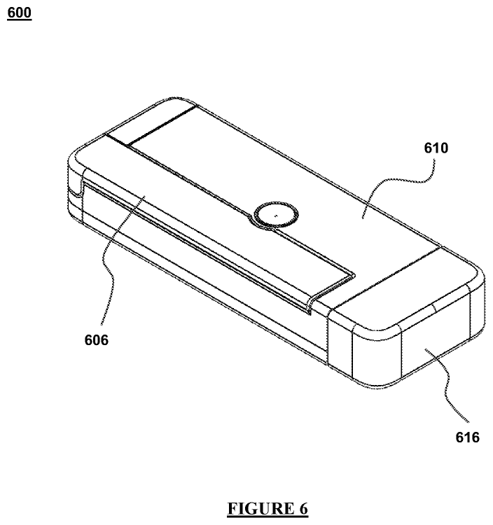

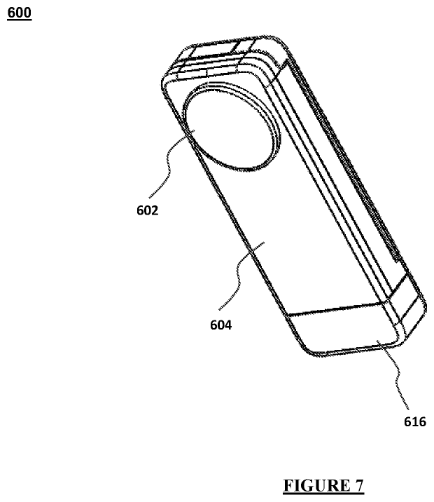

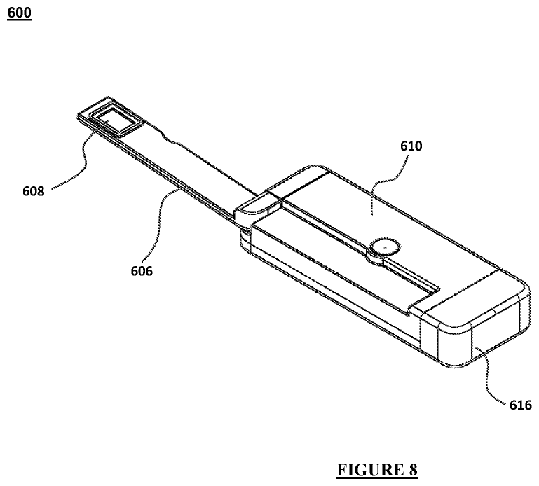

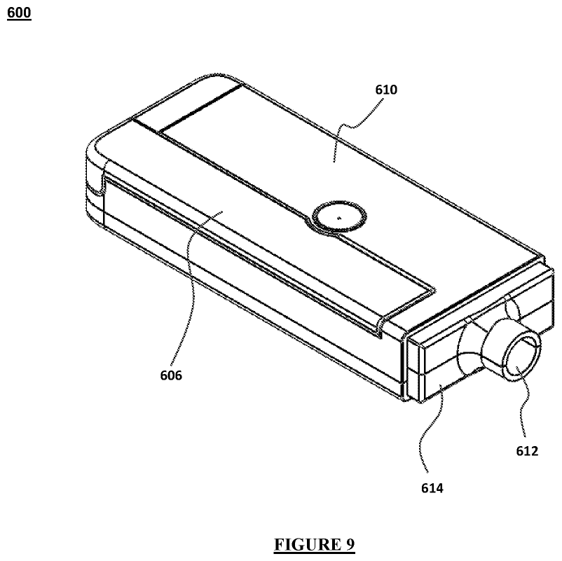

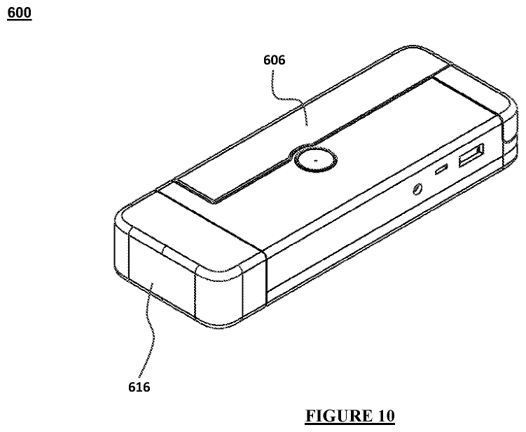

FIGS. 6 to 10 illustrate different perspective views of the integrated medical examination device 600 in accordance with an embodiment of the present subject matter. The integrated medical examination device 600 of the present invention comprises a plurality of components. For example and by no way limiting the scope of the present subject matter, the integrated medical examination device 600 includes an acoustic measurement unit or sound acquisition sensor 602 provided on a first wall 604 of the device 600 for auscultation or listening to internal sounds of the pet animal. According to the present subject matter, the internal sounds of the pet animal can be assaulted or listened by applying the acoustic measurement unit 602 at various predetermined points on the silicon sleeve when worn by the pet animal. The integrated medical examination device 600 also includes a foldable SPO2 measurement unit 606 comprising a foldable SPO2 measurement member 608 that can be brought in contact with one of the lips of the pet animal for measuring SPO2 readings. In an embodiment, the foldable SPO2 measurement unit 606 is rotatably located on a second wall 610 of the integrated medical examination device 600 that is opposite to the first wall 604. The SPO2 measurement member 608 in folded position is shown in FIGS. 6, 7, 9 and 10 and in unfolded position, it is shown in FIG. 8 in an embodiment. Further, the integrated medical examination device 600 includes a temperature measurement unit 612 that is provided at a side wall 614 of the device 600 and can be placed on the wall of the anal region of the pet animal for measuring temperature of the pet animal. In an embodiment, the temperature measurement unit 612 can be covered by a side cover 616, as shown in FIGS. 6, 7, 8 and 10.

Heart and lung auscultations of the pet animal are captured by using the sound acquisition sensor 318. In an embodiment, the sound acquisition sensor 318 may include but is not limited to a microphone capable of acquiring sound data. The sound acquisition sensors 318 are configured to accommodate multiple sound frequencies that are adjusted to fit recording of specific organ sound (as, for example, heart sound frequencies are different than lung sound frequencies). In an embodiment, the device 600 according to the present subject matter includes other aspects such as but not limiting to noise cancellation filters and active noise cancellation mechanisms integrated with products. They further include sound examination peripherals 316. The sound examination peripherals 316 for example, are made of plastic, rubber, silicone, or stainless steel and can be attached to the acoustic housing.

Generally SPO2 is measured only in rare cases in dogs and mainly with anesthetized dogs. This is because of the extreme difficulty in measuring the dog's oxygen saturation levels and pulse in the areas like the tongue and lips and inaccurate readings due to continuous movement in dogs. The present device 600 with its design and by incorporating and eliminating motion artifacts makes it convenient for a pet owner to take the oxygen saturation level for any dog at home.

Due to conditions of fur and remote possibility of standardisation across breeds of dogs, temperature measurement has always been invasive in the past. Conventionally, there are a few aural thermometers. However, such aural thermometers are not always suitable for home use considering that such thermometers have to be placed close to the ear drum. Also, the presence of hair, wax and other debris makes it difficult to accurately take the temperature. In the present device 600, an infrared (IR) based thermometer is employed to take the temperature at the anal verge area of the pet animal. The temperature measurement is specifically calibrated to be used in different breeds of dogs and irrespective of fur and other aspects which generally prevent taking of temperature non-invasively.

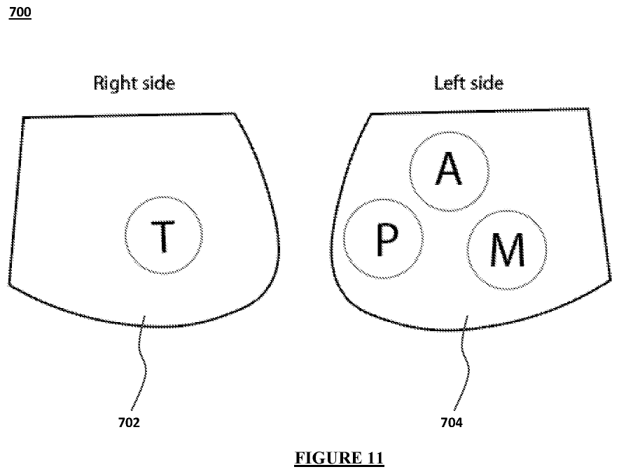

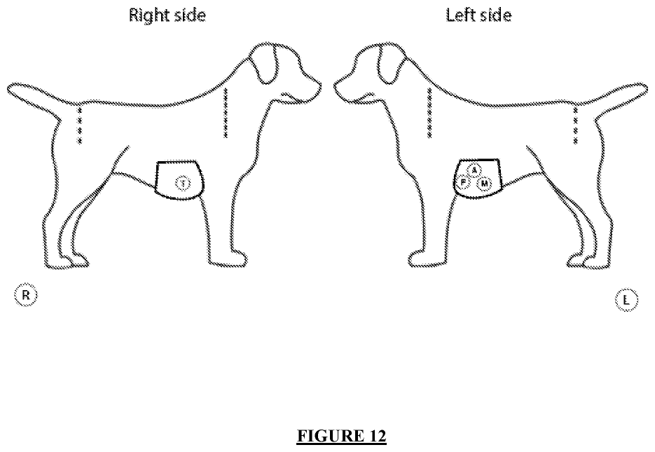

Correct positioning becomes extremely important while taking vitals of a pet animal. For this purpose, the present device has specific peripherals for assistance in taking each of the vital parameter. For the heart and lung auscultation, the present device 600 is accompanied with a silicone like stencil 700, as shown in FIGS. 11 and 12. FIG. 11 illustrates a front view of a pair of silicon stencils in accordance with an embodiment of the present subject matter. FIG. 12 illustrates the pair of silicon stencils mounted on a pet in accordance with an embodiment of the present subject matter. As shown herein, in an embodiment, the stencil 700 comprises a pair of stencil members 702, 704, which can be placed on the chest of the dog to allow the pet owner to correctly identify the various points of recording the heart and lung auscultations. For the temperature measurement, the device 600 has an extra attachment to correctly place it on the anal verge area. For SPO2, the structure of the device 600 takes of the correct positioning of measuring SPO2, in the lips of the dog.

As shown in FIGS. 11 and 12, in an embodiment, the left side of the silicon stencil or left silicon stencil member 704 comprises three predetermined points or cut-outs, i.e., P, A, M and the right side of the silicon stencil or right silicon stencil member 702 comprises one predetermined point or cut-outs, i.e. T, where P is the pulmonary point at which S1 and S2 sounds of the heart can be experienced, A and M are the aortic point and mitral point respectively at which murmurs in the heart can be heard and T is the tricuspid point. The points P, A, M, T define the areas of heart auscultation once the left side of the silicon stencil or left silicon stencil member 704 is placed over the first teat of the pet.

Therefore, the hand-held independent device 600 according to the present subject matter is configured to measure the heart and lung auscultation, core body temperature and pulse and blood oxygenation levels. The device 600 is accompanied by a supporting stencil kind of a silicon sheet structure to enable even pet parents at home to accurately position the device for measure heart and lung auscultations at points P, A, M, T (Mitral area, Tricuspid area, Pulmonary area, Aortic area). The device 600 according to the present subject matter is a single integrated device, with attachments for each measuring each vital parameter. A feedback system is provided to inform the pet parent, whether the reading taken is accurate and whether the reading has to be taken again. The present device is an active device, which is hand-held and gives immediate results.

While the preferred embodiments of the present invention have been described hereinabove, it should be understood that various changes, adaptations, and modifications may be made therein without departing from the spirit of the invention and the scope of the appended claims. It will be obvious to a person skilled in the art that the present invention may be embodied in other specific forms without departing from its spirit or essential characteristics. The described embodiments are to be considered in all respects only as illustrative and not restrictive.

1. A health and wellness system for an animal, such as a pet, the health and wellness system comprising:
a portable examination device configured to perform one or more aspects of examination of the animal in a non-invasive manner, the portable examination device comprising one or more examination units for obtaining and recording essential vitals of the animal by a user; and
a processing unit for storing and managing data corresponding to the essential vitals and medical information of the animal.

2. The health and wellness system of claim 1, wherein the essential vitals comprise one or more of core body temperature of the animal, heart auscultations, lung auscultations as well as pulse and oxygen saturation.

3. (canceled)

4. The health and wellness system of claim 1, further comprises a silicon stencil to correctly position the portable examination device on body of the animal and obtain heart and lung auscultations at different positions.

5. The health and wellness system of claim 1, further comprises a feedback system for providing feedback about correctness of data acquired by the portable examination device to the user.

6. (canceled)

7. The health and wellness system of claim 1, wherein the one or more examination units of the portable examination device comprise one or more heat based sensors, one or more acoustic sensors, one or more photo optic sensors and one or more motion sensors.

8. The health and wellness system of claim 7, wherein the one or more acoustic sensors are mounted on a first wall of the portable examination device and is configured to sense sound from heart and lung area of the animal, and wherein the one or more acoustic sensors comprise non-contact reflective acoustic units that are configured to cancel external noise while recording the heart and lung sounds.

9. (canceled)

10. The health and wellness system of claim 7, wherein the one or more heat based sensors comprise temperature sensors having one of infrared (IR) temperature sensors to calculate the core body temperature of the animal.

11. The health and wellness system of claim 10, wherein the one or more temperature sensors are mounted on a side wall of the portable examination device and are configured to obtain temperature reading of the animal merely by getting in contact with anal verge area of the animal.

12. The health and wellness system of claim 7, wherein the one or more photo optic sensors comprise a foldable SPO2 measurement unit having a foldable SPO2 measurement member for measuring SPO2 readings of the animal merely by contacting the foldable SPO2 measurement member with one of lips of the animal.

13. The health and wellness system of claim 7, wherein the one or more motion sensors are configured to evaluate the accuracy of the readings and to give feedback and guidance to the user for accurate measurement.

14. The health and wellness system of claim 7, wherein the one or more motion sensors comprise spatial motion sensors having a processing engine to measure change of position of the animal over time to yield physical parameters.

15. The health and wellness system of claim 1, further comprises a filtering circuitry to capture heart and lung auscultations of the animal.

16. The health and wellness system of claim 1, further comprises a noise handling mechanism to obtain pure data for heart rate.

17. The health and wellness system of claim 1, wherein the processing unit comprises a pet data repository for storing data corresponding to the essential vitals of the animal recorded by the portable examination device, storing medical history and other related information of the animal, and a management system for managing the data of the animal.

18. The health and wellness system of claim 1, wherein the processing unit further comprises a pet owner module and a veterinarian module connected with each other, wherein the pet owner module comprises a memory unit and a processor embedded in the medical examination device, and wherein the veterinarian module comprises a trained reference medical data repository.

19. (canceled)

20. The health and wellness system of claim 18, wherein each of the pet owner module and the veterinarian module comprises a video streaming module for enabling video streaming of the user with a medical professional.

21. (canceled)

22. The health and wellness system of claim 1, wherein the processing unit comprises a first interface for the user that provides generic health of the pet with an alert system to enable the user to connect with a medical professional and a second interface for the medical professional that provides real time data about the animal on all the essential vitals.

23. A health and wellness system for an animal, comprising:
an examination module configured to capture sensor data corresponding to essential vitals of the animal from one or more sensors;
a pet data repository for storing sensor data;
a microcontroller configured to obtain the sensor data as input and process the sensor data; and
a wireless fidelity based high speed radio module configured for direct data communication with a remote cloud server.

24. The health and wellness system of claim 23, wherein the pet data repository comprises a data storage system configured to store various data relating to the animal acquired by the one or more sensors and various medical data acquired during medical examination.

25. The health and wellness system of claim 23, further comprises a battery source voltage level measurement module configured to measure voltage level of the battery source.
